(19) United States
(12) Patent Application Publication
(10) Pub. No.: US 2025/0051706 A1
(43) Pub. Date: Feb. 13, 2025

(54) MODIFIABLE HYDROGEL MATERIAL AND METHOD FOR PRODUCING A MODIFIABLE HYDROGEL

(71) Applicant: LEIBNIZ-INSTITUT FÜR POLYMERFORSCHUNG DRESDEN E. V, Dresden (DE)

(72) Inventors: Elisha KRIEG, Dresden (DE); Yu-Hsuan PENG, Dresden (DE); Krishna GUPTA, Dresden (DE); Syuan Ku HSIAO, Dresden (DE)

(21) Appl. No.: 18/719,634

(22) PCT Filed: Dec. 21, 2022
(86) PCT No.: PCT/DE2022/100974

§ 371 Date: Jun. 13, 2024

(30) Foreign Application Priority Data
Dec. 22, 2021 (DE) 10 2021 134 240.6

(51) Int. Cl. C12M 1/12; C12N 5/00

(52) U.S. Cl. CPC C12M 25/02; C12N 5/0062; C12N 2533/20; C12N 2533/30; C12N 2537/10

(57) ABSTRACT

A modifiable hydrogel material has main chain polymers which are modified with anchor modules in the form of predetermined functionalized single strands of DNA. The main chain polymers can be crosslinked with one another by intermolecular DNA double strand formation. A DNA sequence of the anchor modules has a predetermined number of specific sequence positions N with different base combinations and/or is blocked by a temperature-dependent DNA blocking strand. In a method for producing a hydrogel with the modifiable hydrogel material, DNA modules in the form of free DNA single strands or DNA modules in the form of predetermined DNA single strand pairs which bind to anchor modules in a complementary manner are employed for crosslinking the main chain polymers, which form an intermolecular DNA double strand at a common binding domain.

The invention relates to a modifiable hydrogel material and to a method for producing a modifiable hydrogel. The hydrogel material is intended in particular for use as a matrix for 3D cell cultures and organoid structures.

Hydrogels describe gels of water-soluble polymers which can bind water. In biotechnology, three-dimensional structures of hydrogels are employed as cell culture matrices in order to allow the cultivation of living cells outside an organism. In contrast to two-dimensional surfaces, such as, for example, classical Petri dishes, three-dimensional cell culture matrices can better simulate the properties of biological tissue and thus reproduce a nature-faithful environment or physiological environment for optimal cell growth. This makes it possible to investigate biological issues and biochemical relationships in vitro, to analyse disease progressions and to develop individual therapy methods. With the help of organ-like multicellular structures, so-called organoids, the function and development of human organs can be modelled, drugs tested, and finally replacement organs from the patient's own cells be produced. Generally, three-dimensional cell culture matrices can be divided into two categories. The first category comprises cell culture matrices of biological origin. The second category comprises chemically defined cell culture matrices. So far, the growth of organoids has been almost exclusively in materials of biological origin. Usually, reconstituted basement membrane-like matrices are employed, such as the product Matrigel (from Corning Life Sciences, New York, USA). Matrigel is a complex protein-containing mixture obtained from a cancer cell line of mice.

It has been shown that the field of application of biologically created cell culture matrices is restricted. Thus, there are no possibilities for adapting or changing the material properties of organoid structures produced from Matrigel. Further, the composition, which is not precisely defined, shows significant batch-to-batch variations. Also, undesirable components of Matrigel can only be removed or alternatively replaced with great effort. Finally, because of the biological origin of an application of such organoid structures based on Matrigel for regenerative medicine, it is considerably restricted.

There is a great need for such restrictions to be removed and for hydrogels having a defined synthetic composition to be created for the three-dimensional cell culture. This is associated with the object of providing a material which possesses the ability to adapt the composition, porosity and mechanical properties to the needs of cells and/or to change them dynamically in a predetermined manner. Further, the material should be capable of being applied under cell-compatible conditions in order to be able to be processed into a three-dimensional structure in the application of an additive method, such as 3D printing, for example.

It is therefore the object of the invention to provide a hydrogel material with which the restrictions known from the prior art can be eliminated, in particular with regard to the use of the material as an organoid structure.

The object is achieved by a subject matter having the features according to patent claim 1. Further developments are indicated in the dependent claims.

The invention is based on the knowledge that stabilising crosslinks of hydrogels can be achieved by DNA molecules which impart to the material specific viscoelastic, kinetic and thermodynamic properties which can be predicted by a specific DNA sequence design and computer-assisted methods, and can be dynamically changed by adding further DNA components.

A hydrogel material having main chain polymers is proposed, wherein the main chain polymers are modified with anchor modules in the form of predetermined functionalised DNA single strands. According to the invention, the main chain polymers can be crosslinked with one another by intermolecular DNA double strand formation, wherein a DNA sequence of the anchor modules has a predetermined number of specific sequence positions N with different base combinations and/or is blocked by a temperature-dependent DNA blocking strand. The number n of the N-bases within the DNA sequence defines the number of anchor modules differing in sequence within the hydrogel material. Preferably, the number n of the sequence positions N is limited to a maximum of 5. The DNA sequence of the DNA single strands of the anchor modules can preferably contain 14 to 60 nucleotides. The temperature-dependent DNA blocking strand is a DNA sequence which at least in sections binds in a complementary manner to the DNA sequence of a DNA single strand of an anchor module in order to block a binding site on the anchor module in order to form the crosslink. A hydrogel produced from the hydrogel material thus has crosslinks of the main chain polymers based on DNA molecules. The main chain polymers can additionally be modified with peptide side chains. These peptide side chains contain one or more adhesion motifs which enable the binding of cells to the matrix. Such adhesion motifs are, for example, the RGD or IKVAV peptide sequences. Accordingly, the main chain polymers can have peptide side chains with the RGD sequence and/or with the IKVAV sequence.

The structure of the main chain polymers is preferably by free radical chain reaction of acryloyl-based monomers. Derivatives of acrylamide and acrylic acid and mixtures thereof in any ratios can be employed as monomers, for example. The main chain polymers can thus be formed from derivatives of acrylamide monomers and acrylic acid monomers. By using small amounts of initiator, such as ammonium persulphate, molecular weights of the main chain polymers of from 100 kDa to 50 MDa can be achieved with the exclusion of oxygen. The main chain polymers of the hydrogel material can thus each have a molecular weight in the range of 100 kDa and 50 MDa.

In principle, all hydrogel-forming polymers which permit covalent binding of anchor modules in the form of predetermined functionalised DNA single strands can be employed as main chain polymers.

According to the invention, the main chain polymers are modified with functionalised DNA single strands, the anchor modules. The DNA single strands fulfil the function of side chains which are covalently bonded to the main chain polymers. In the simplest case, crosslinking can be achieved by intermolecular DNA double strand formation of DNA single strands of the anchor modules of adjacent main chain polymers. In this case, the anchor modules can have a common binding domain for forming the crosslink and, in addition, at least one further binding domain. Apart from the desired crosslink by double strand formation with DNA single strands of further main chain polymers, further functions can be assigned to the DNA single strands of the anchor modules. For example, the DNA single strands of the anchor modules can have covalent modifications. For example, a modification of the DNA single strands in the form of modified bases, for example methylated cytosine, or a modified phosphate sugar backbone, for example phosphorothioate, can be provided.

The hydrogel material according to the invention can furthermore contain DNA modules in the form of free functionalised DNA single strands which are provided for intermolecular interaction with the anchor modules. These DNA modules, which are also referred to below as splint, consist of free synthetic DNA single strands which can integrate into the hydrogel material by molecular self-assembly, for example by interaction with the anchor modules, and impart specific properties to the hydrogel material.

The DNA modules have a predetermined anchor module binding domain, which enables them to bind to a correspondingly complementary domain on the DNA anchor modules. Free single strands of the DNA modules thus serve as crosslinkers in that they connect the DNA single strands of anchor modules of different main chain polymers to one another by intermolecular DNA double strand formation. The DNA modules are therefore preferably employed for forming the crosslinks.

The number of intermolecular links in the hydrogel material is directly related to the mechanical strength. The efficiency of crosslinks between the main chain polymers can be impaired by intramolecular binding, in particular at low concentrations. In order to overcome this problem, DNA modules in the form of complex crosslinker libraries (CL) consisting of pairs of DNA single strands which have a common binding domain and in each case an anchor module binding domain for binding to the anchor modules of the main chain polymers are used. In the region of the common binding domain, the DNA single strand pairs, which can also be referred to as splint pairs, connect under appropriate conditions to form a DNA double strand. The high potential binding specificity of the DNA is utilised by the DNA single strand pairs or splint pairs in order to suppress the occurrence of intramolecular crosslinks and thus promote intermolecular crosslinking. It can thus be provided that DNA single strand pairs (split pairs) predetermined as DNA modules are employed. These DNA single strand pairs have a common binding domain in order to form a DNA double strand. The common binding domain is a region of the DNA sequence in which the two splints of a splint pair connect to one another in a sequence-specific manner. Also, each DNA single strand of the split pairs has an identical anchor module binding domain which binds to the anchor module of a main chain polymer and forms an intermolecular DNA double strand.

The anchor module binding domain and the common binding domain of the DNA modules can have different melting temperatures (T1, T2), wherein the melting temperature T1 of the anchor module binding domain is higher than the melting temperature T2 of the common binding domain of the split pairs. As a result, the splints, i.e. the free DNA single strands, initially bind to the DNA single strand of the anchor module at a melting point T1 during slow cooling of, for example, 80° C. As soon as the temperature reaches the lower melting point T2, the binding domains of the DNA modules begin to bind with their suitable partners. The common binding domains can either be designed such that their sequences are orthogonal to one another, i.e. the sequences are unique and optimised for maximum binding specificity (Orthogonal Crosslinker Libraries, OCL). Alternatively, the libraries can also be constructed by combinatorial methods, such as by introducing different base combinations at specific positions N of the sequence of the common binding domain. A corresponding library containing splint pairs is referred to below as CCL (Combinatorial Crosslinker Libraries, CCL). At a sequence position N, the sequence can have one of the four bases of guanine (G), cytosine (C), thymine (T) or adenine (A). The probability that a DNA strand has one of the bases at one of the sequence positions N is as a rule 25% for G, 25% for C, 25% for T, and 25% for A. Ratios of the bases C, G, A, T deviating therefrom are conceivable. The number n of N-bases within the binding domain determines the number of splint pairs in a CCL. Thus, the complexity or size of a CCL increases with formula 4″: For example, a CCL with n=1 contains 4 splint pairs; a CCL with n=2 contains 16 splint pairs and a CCL with n=3 contains 64 splint pairs.

The hydrogel material according to the invention can thus contain DNA single strand pairs, i.e. splint pairs, which are selected from a library containing DNA single strand pairs which differ in that the DNA sequence of the common binding domain has at least one different base combination at a specific sequence position N. The number of splint pairs contained in the library depends on the formation rule with respect to the number n of the specific sequence positions N. The hydrogel material according to the invention can thus contain DNA single strand pairs, i.e. splint pairs, which are selected from a library containing DNA single strand pairs in which the DNA sequence of the common binding domain has a predetermined number n of specific sequence positions N with different base combinations, wherein the number n is in the range from 0 to 5.

Statistical simulations and rheological experiments prove that ineffective intramolecular crosslinks can be suppressed by using CLs (OCLs or CCLs). As a result, effective intermolecular crosslinks are increasingly formed between the main chain polymers, which increases the elasticity of the hydrogel material and reduces the amount of DNA required for gelling the hydrogel material. In addition, the elasticity can be controlled by adjusting the CCL complexity, which represents a possibility for adjusting the mechanical properties of the hydrogel material.

The hybridisation of two complementary DNA single strands (ssDNA molecules) of the DNA modules is generally carried out at a high reaction rate. Such a rapid and reversible binding enables so-called self-healing properties and facilitates application of the hydrogel material through a nozzle, such as during printing. If cell-compatible conditions are required, however, the rapid reaction kinetics can also pose a challenge in the self-assembly of the hydrogel material. If the main chain polymers are mixed with the DNA modules and cells, the rapid crosslinking results in incomplete mixing of the hydrogel material. The resulting hydrogel material then has a heterogeneous structure, which is associated with a loss of strength. This problem is solved with temperature-dependent blocking strands. These are DNA single strands which block the binding sites of the DNA modules and/or of the anchor modules at low temperature, i.e. below the melting temperature, by binding. Thus, the different components of the hydrogel material, in particular cells and medium, can be optimally mixed at low temperature. In order to initiate the gelling of the hydrogel material, i.e. to start the crosslinking of the hydrogel material, the temperature must be increased first. For this, the sequence of the blocking strands is selected, for example, such that they spontaneously dissociate from the anchor module or from the DNA module at a temperature of 15° C. The binding sites released or freed as a result of the release of the blocking strands then permit rapid crosslinking. It can thus be provided that the DNA modules are blocked by a DNA blocking strand at a temperature below a predetermined dissociation temperature. The sequence of the blocking strands can be selected such that the dissociation temperature is in the range from 4° C. to 37° C. Advantageously, a corresponding application of blocking strands leads to a significantly higher stiffness and a higher homogeneity of the hydrogel material.

The rate of crosslinking can furthermore be influenced by reversible structure-forming DNA sequences of the anchor modules and/or of the DNA modules. Thus, the DNA single strands can have sequences which form a reversible hairpin loop structure below their melting temperature. In this structure, a DNA single strand forms an intramolecular DNA double strand by binding a part of the single strand to itself in a complementary manner. In this way, self-blocking can be achieved. Part of the blocking sequence can thereby form part of the common binding domain of the splint pairs. Furthermore, it can be provided that the blocking sequence is formed as a part of the anchor module binding domain.

It can thus be provided that the anchor modules and/or the DNA modules have structure-forming sequences which slow down the intramolecular crosslinking within the hydrogel material due to their self-blocking properties. Due to the fact that anchor modules and/or DNA modules are employed in this variant which form a self-blocking hairpin loop structure, no separate free blocking strands are advantageously required. The hairpin loop structure as a secondary structure slows down crosslinking, since the respective binding domains must be released for crosslinking by spontaneous opening of the hairpin loop structure. This application has the advantage that no blocking strands are released into the medium as a result of the release of the hairpin loop structure.

It has been shown that a stress relaxation behaviour of the hydrogel material according to the invention can be influenced by varying the base sequence and the sequence length of the anchor module binding domain and/or the base sequence and the sequence length of the common binding domain. On the one hand, the stress relaxation behaviour of the hydrogel material according to the invention can be adjusted on the basis of the sequence length of the anchor module binding domain and/or on the basis of the sequence length of the common binding domain of the DNA modules. On the other hand, such changes in the stress relaxation behaviour can also be achieved by changing the base arrangement within sequences of the same length. These DNA modules, also referred to as stress relaxation crosslinkers (SRCs), thus comprise several DNA single strands which have different nanomechanical binding strengths by systematic change of the base sequence or length of the binding domain. With the help of SRCs, the stress relaxation behaviour of the hydrogel material can be set systematically over a wide range, wherein stress relaxation times (τ) of τ>0.01 s to τ<10000 s can be reached. The special feature of SRCs is that, unlike in the methods described up to now (for modifying the stress relaxation behaviour), they enable adjustment of the stress relaxation times τ independently of the elasticity and viscosity, as well as without changes in other chemical properties of the material or of the medium. Preferably, the sequence of the anchor module binding domain and/or of the common binding domain has 8 to 22 nucleotides.

According to an advantageous further development, the hydrogel material according to the invention can have bait DNA in the form of free predetermined synthetic DNA single strands or double strands. These synthetic DNA molecules are added to the buffer system or to the medium. Released bait DNA molecules can serve as an alternative (sacrificial) substrate for nucleases. Free-dissolved DNA molecules have the task of increasing the service life of other functional DNA modules in the hydrogel material. It has been shown that the material properties of the hydrogel material are not influenced or only slightly influenced by the bait DNA degradation. Bait DNA molecules can be composed of regular oligonucleotides. In order to increase their stability, a synthetic modification, for example with phosphorothioate backbone, can further be provided.

The hydrogel material according to the invention can contain cells which can proliferate and differentiate within the structure of the hydrogel material. As a result of the cell growth, nucleases such as DNase I may be secreted by the cells into the matrix of the hydrogel material. In addition, nucleases can be present in commercially available serums which can be a constituent of the hydrogel material. This can be disadvantageous since the DNA-based crosslink of the hydrogel material can be attacked by the nucleases. In order to improve the long-term stability of the hydrogel material, the activity of nucleases can be suppressed with the help of the protein actin. It can therefore be provided that the hydrogel material has a predetermined proportion of the protein actin. Alternatively, other nuclease inhibitors, such as, for example, gentamicin and/or rutin, can be employed. Furthermore, the activity of nucleases can be inhibited by employing chelators, such as citrate, for example. As a result, chelators, in particular citrate, may alternatively or additionally be present as a constituent in the hydrogel material or may be added separately. By using inhibitors or by using nuclease-free cell culture media, the hydrogel material is stable over several weeks and can thus be employed for long-term cell cultures and organoid studies.

If enzymatic degradation or degradation of the hydrogel material according to the invention is desired, the rate of enzymatic degradation can be effectively adjusted by adding nucleases and their inhibitors. By employing nucleases in a targeted manner, for example, the stiffness of the crosslinked hydrogel material can be changed and cells released under mild conditions.

It can be provided that the anchor modules and/or the added DNA modules have at least one modified DNA domain with functionalisation as a DNA switch, DNA sensor, DNA enzymatic actuator and/or an aptamer. DNA sequences in the form of shear-loop sequences or zipper-loop sequences which, in combination with FRET detection, can make visible mechanical stresses in the material in the fluorine microscope are conceivable as DNA switches, for example. Furthermore, using DNA-modifying enzymes such as polymerases, exonucleases, restriction enzymes, helicases, ligases or transcriptases, a structural change of the anchor modules and/or the DNA modules can be caused.

In the hydrogel material according to the invention, the binding strength and specificity, binding kinetics, nanomechanical properties, and stimulus responsiveness can be predetermined by the DNA sequence design of the anchor modules and the DNA modules. In this way, mechanical properties can be precisely predefined and dynamically changed. Furthermore, the hydrogel material according to the invention has switchable viscoelastic properties and can be extended by sensory functions which are executed by appropriately functionalised DNA modules. The hydrogel material according to the invention can advantageously be applied by printing in an additive method and can thus be employed as a bio ink.

The hydrogel material can preferably be employed as a cell culture matrix, so that complex chemo-mechanical interactions of cells, tissues and organoids with their environment can be simulated, observed and dynamically changed. In this case, the stress relaxation of the hydrogel material can be adapted without changing the biochemical properties of the basic structure or of the medium.

An essential advantage is the modular structure of the hydrogel material with regard to the functional extensibility by the use of the DNA modules, which can be bound to one or more main chain polymers in any combination by sequence-controlled self-assembly. In this case, there is always an adaptation possibility in that the hydrogel material is extended by additional further DNA modules which can have different functionalisations. For example, a three-dimensional cell culture matrix can be extended with nanomechanical stress sensors after cell compatibility testing is completed. Bound DNA modules can be activated, modified, deactivated, or released again as needed.

Due to the high molecular weight of the main chain polymers, a high crosslinking efficiency can be achieved, as a result of which only small amounts of DNA are required overall in order to achieve the crosslink. As a result, the hydrogel material according to the invention can be produced comparatively cost-effectively. At the same time, the risk of stimulation of unwanted inflammatory reactions of the cells is reduced by the low DNA concentration. In fact, the hydrogel material according to the invention is distinguished by very low stimulation of immune reactions, which is comparable to reference materials employed clinically, such as polytetrafluoroethylene (PTFE). Also, the hydrogel material is hemo-compatible and induces significantly less coagulation in human blood than PTFE or glass-based reference materials.

The defined composition of the hydrogel material according to the invention from purely synthetic components improves reproducibility and reduces regulatory hurdles for commercial applications in the field of medical technology. The main chain polymers and the DNA modules can be filtered sterile and can be stored at a temperature of −20° C. almost without limit. In lyophilised form, they are stable for years even at room temperature (20° C.). Commercial kits of the hydrogel material can thus be stored and transported in an uncomplicated manner.

The advantages of the hydrogel material according to the invention can further be summarised as follows:
The hydrogel material is essentially non-cytotoxic and supports the adhesion of biological cells by covalently bound peptide adhesion motifs.
The synthetic DNA modules contained in the hydrogel material define the properties in a predictable manner.
The hydrogel material can be extended modularly under cell-compatible conditions by self-assembly.
The hydrogel material has an adjustable stiffness.
The hydrogel material has an adjustable stress relaxation time.
The hydrogel material has adjustable crosslinking kinetics.
The adaptation of the different properties is generally orthogonal to one another, i.e. properties can be modulated independently of one another.
The hydrogel material is printable by extrusion and can be employed as a bio ink.

As an alternative to the modular self-assembly with anchor modules, all DNA modules described here can also be covalently bonded directly to the main chain polymers. Although such direct binding limits the modularity of the material, it can be used to reduce material costs or to exclude the release of DNA even under denaturing conditions.

The hydrogel material is suitable as a viscoelastic matrix for a broad spectrum of cells and organoids. In particular, it has a high viability and proliferation of mesenchymal stem cells (MSC), Madin-Darby canine kidney cells (MDCK), pluripotent stem cells (PSC), and trophoblasts. Further, it promotes morphogenesis and the formation of organoids, such as human placenta organoids.

The invention includes modifiable hydrogels based on the modifiable hydrogel material. The modifiable hydrogel material is provided for producing a modifiable hydrogel. Furthermore, the invention includes all methods for modifying the hydrogel material which result from the field of application and the features of the hydrogel material according to the invention. The modification of stress relaxation behaviour is to be mentioned as an example. Further modification possibilities result from the described functionalisations of the DNA modules.

The invention further relates to a method for producing a hydrogel with a hydrogel material which has main chain polymers in its basic configuration, which are modified with anchor modules in the form of predetermined functionalised DNA single strands, wherein DNA modules in the form of free DNA single strands or DNA modules in the form of predetermined DNA single strand pairs which bind to the anchor modules in a complementary manner are used for crosslinking the main chain polymers to the anchor modules, which form an intermolecular DNA double strand on a common binding domain, wherein each DNA single strand has an identical anchor module binding domain which binds to the anchor module of a main chain polymer and forms an intermolecular DNA double strand. For crosslinking, the DNA modules are brought into contact with the main chain polymers.

In order to influence the crosslinking and to further modify the hydrogel material or the hydrogel, it can be provided that temperature-dependent DNA blocking strands are employed which bind to the anchor modules and/or the DNA modules and block crosslinking of the hydrogel material at a temperature below the dissociation temperature of the DNA blocking strands. The crosslinking kinetics are influenced by the use of temperature-dependent DNA blocking strands, which gives the hydrogel material or the hydrogel specific properties.

Furthermore, it can be provided that DNA modules which have a structure-forming DNA sequence which block themselves below a dissociation temperature and form a hairpin loop structure are employed for the production of modifiable hydrogels. In this embodiment variant, the hydrogel material must be heated to a temperature above the dissociation temperature of the hairpin loop structure in order to initiate the crosslinking process as a result of a release of the common binding domain or of the anchor module binding domain.

For crosslinking the hydrogel material, DNA modules can also be employed in which a DNA sequence of the common binding domain has a predetermined number n of specific sequence positions N with different base combinations at splint pairs, wherein the value of the number n is 1, 2, 3, 4 or 5. Thus, a crosslinked hydrogel material with correspondingly 4, 16, 64, 256 or 1024 different splint pairs can be provided.

Furthermore, DNA modules can be employed for crosslinking, in which the anchor module binding domain and the common binding domain of splint pairs have different melting temperatures (T1, T2), wherein the melting temperature T1 of the anchor module binding domain is higher than the melting temperature T2 of the common binding domain. For crosslinking, a corresponding temperature control of the hydrogel material can be provided.

The sequence(s) of the anchor module binding domain and/or of the common binding domain of the DNA modules employed for crosslinking can have 8 to 22 nucleotides. The number of nucleotides of the sequence(s) of the anchor module binding domain and/or of the common binding domain can influence the stress relaxation behaviour of the hydrogel material or of the hydrogel.

The hydrogel material according to the invention is provided in particular for the production of a hydrogel which is used as a matrix for 3D cell cultures and/or organoid structures.

Further details, features, and advantages of designs of the invention will become apparent from the following description of exemplary embodiments with reference to the accompanying drawings. In the drawings:

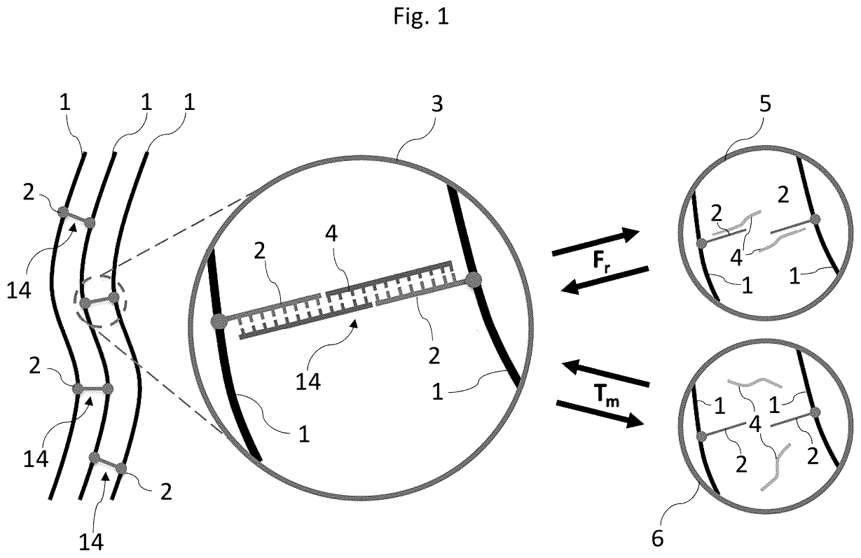

FIG. 1: shows a schematic representation for explaining the structure of the hydrogel material,

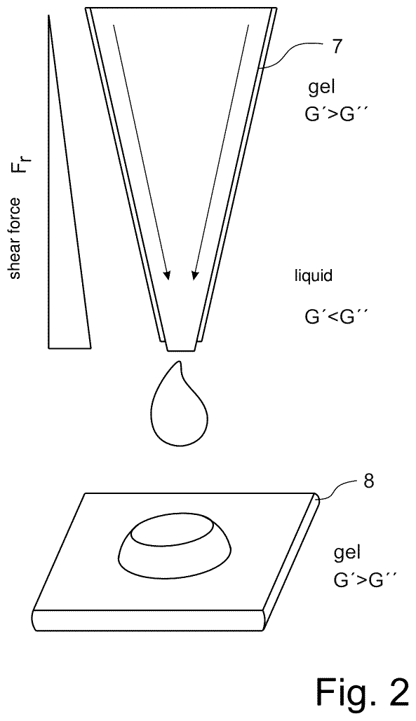

FIG. 2: shows a schematic representation of an example for the application of the hydrogel material,

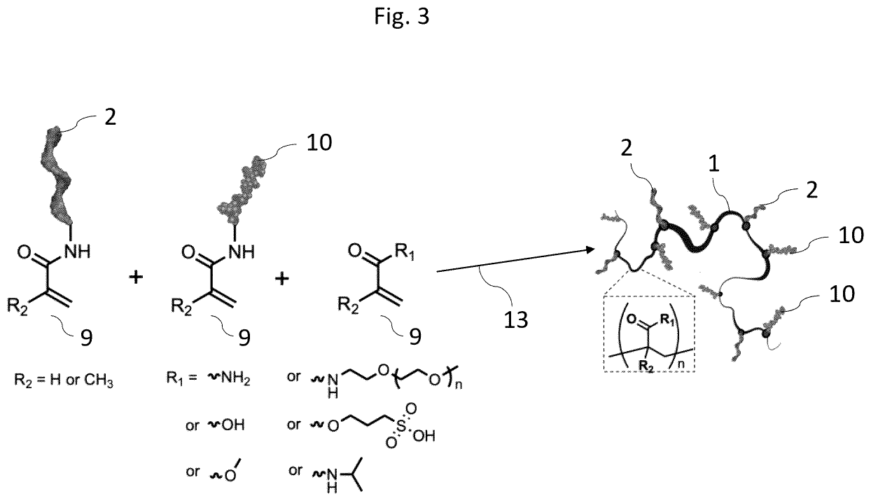

FIG. 3: shows a schematic representation for explaining the structure of the main chain polymers of the hydrogel material,

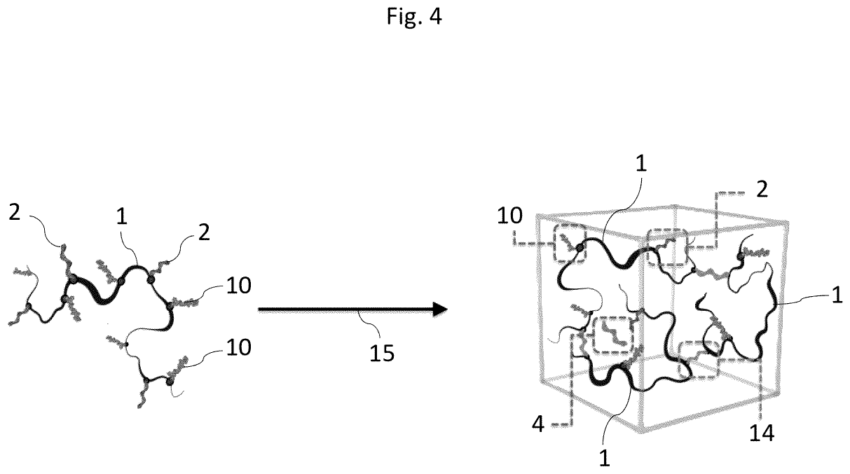

FIG. 4: shows a further schematic representation for explaining the structure of the hydrogel material,

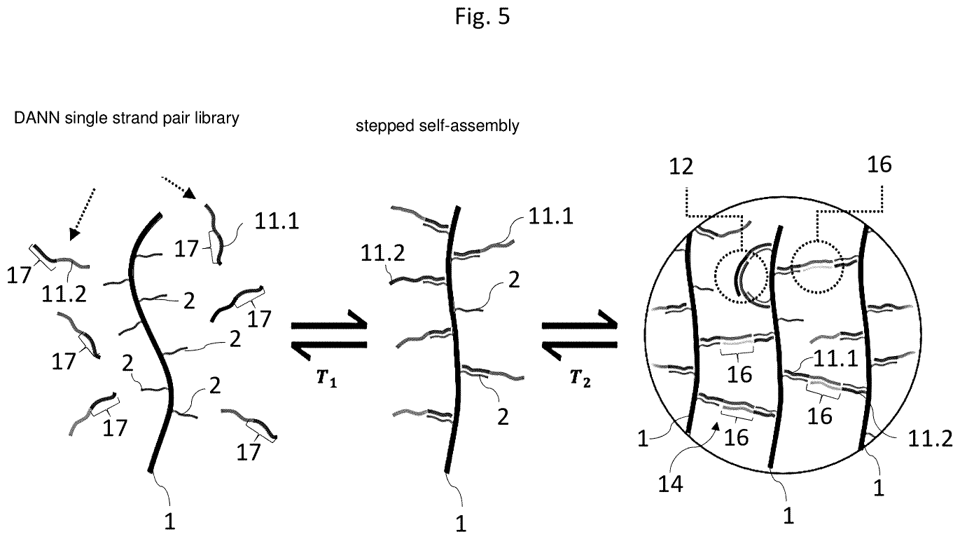

FIG. 5: shows a schematic representation for explaining the crosslinking of the hydrogel material using splint pair libraries,

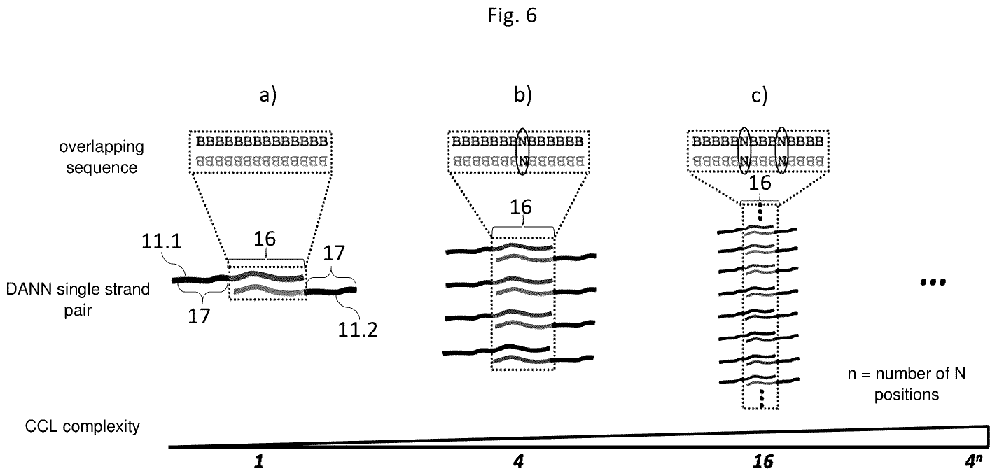

FIG. 6: shows a further schematic representation for explaining the structure of combinatorial splint pair libraries (CCLs),

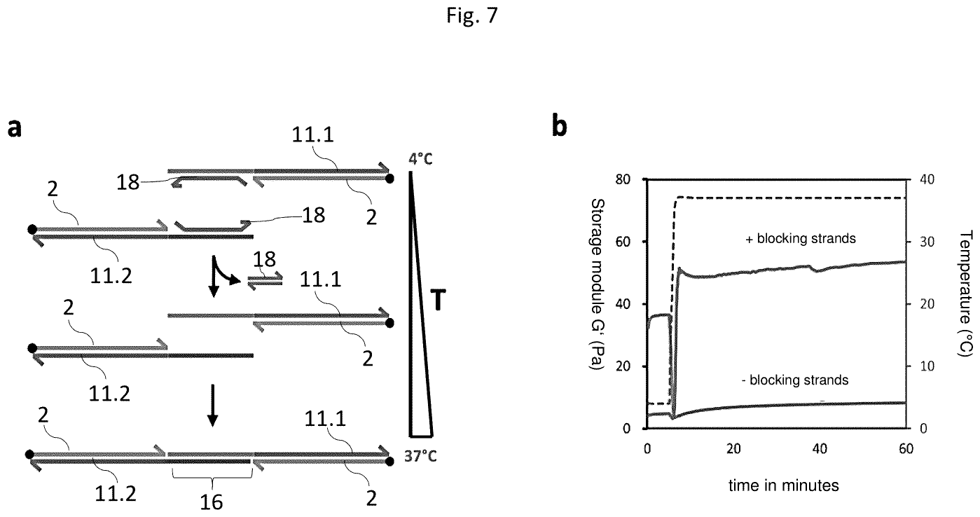

FIG. 7: shows a schematic representation for explaining temperature-dependent DNA blocking strands,

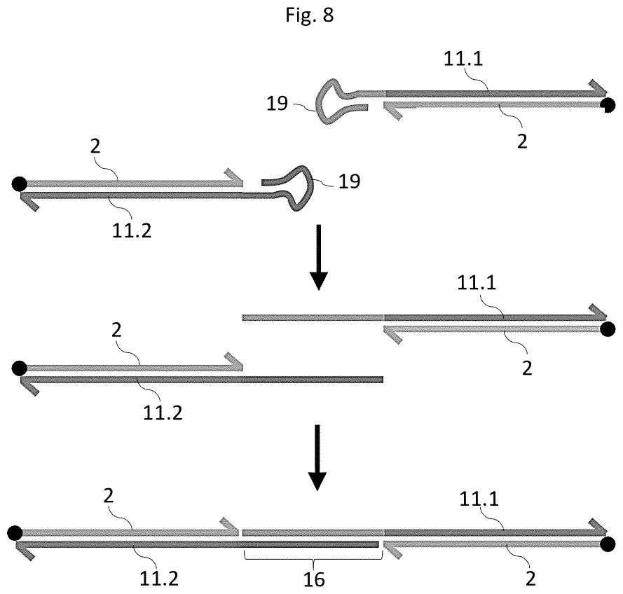

FIG. 8: shows a schematic representation for explaining the structure of DNA modules with the property of temperature-dependent self-blocking.

The DNA sequences represented in the figures serve to illustrate and explain the functioning of certain biochemical functionalisations or modifications of the DNA molecules by way of example.

FIG. 1 shows a schematic representation for explaining the structure of the hydrogel material according to the invention. The hydrogel material has main chain polymers 1 formed from acryloyl-based monomers, which are modified with predetermined peptide side chains (not shown here) and anchor modules 2 in the form of functionalised DNA single strands. The anchor modules 2 form crosslinks 14 between the main chain polymers 1.

An enlargement 3 of a region of the main chain polymers 1 is represented within the circle. This enlargement 3 shows two opposite main chain polymers 1 with the modified DNA single strands of the anchor modules 2 thereon. It can be recognised that the main chain polymers 1 are crosslinked to one another by employing additional DNA modules 4 in the form of DNA single strand pairs as a result of intermolecular DNA double strand formation of the anchor modules 2. The free DNA modules 4 thus serve as crosslinkers. This crosslink 14 is by DNA hybridisation.

The crosslink 14 is achieved by adding the DNA modules 4 and is reversible, as can be seen with the enlargements in the circles 5 and 6. The enlarged representation 5 shows the influence of shear forces Fr on the crosslink 14. If shear forces Fr act on the hydrogel material, the crosslink 14 of the main chain polymers 1 can be dissolved, since the DNA modules 4 are detached from the anchor modules 2 as a result of the mechanical action of force. By reducing the shear forces Fr, the DNA modules 4 can again bind to the anchor modules 2, whereby the DNA double strand and consequently the crosslink 14 can be restored according to the enlargement 3. This is illustrated with the arrows between the enlargements 3 and 5. The enlargement 6 represents the influence of the temperature Tm on the crosslinks 14. An increase in the temperature above the melting point Tm of the DNA crosslinks 14 leads to a dissociation of the DNA double strands, as a result of which the crosslinks 14 of the main chain polymers 1 are dissolved. If the temperature is reduced below Tm, the DNA modules 4 bind again to the anchor modules 2 and form a DNA double strand, whereby the crosslinks 14 are restored. It can be provided that the parameters of shear force Fr and temperature Tm influencing the crosslinks 14 are combined in order to change the properties of the hydrogel material.

As a result of a specific DNA sequence design, the properties of the crosslinks 14, i.e. mechanical and thermodynamic stability, as well as the binding kinetics and the topology of the intramolecular network of the hydrogel material can be predicted and changed. The properties of the DNA modules 4 as crosslinkers thus have a direct influence on the macroscopic material properties of the hydrogel material according to the invention. This relates, for example, to the material properties of melting point and stress relaxation.

FIG. 2 shows a schematic representation of an example for the application of the hydrogel material according to the invention. It has been shown that, in the hydrogel material according to the invention, the binding energy AG, the melting temperature Tm, the speed constants K and the mechanical stability in the DNA base sequences of the DNA single strands of the anchor modules 2 employed here for crosslinking or of the free DNA modules 4, which are employed as crosslinkers, are coded. This ability can be used to achieve shear-thinning and self-healing properties of the hydrogel material according to the invention. Advantageously, the hydrogel material according to the invention is an elastic solid under a low mechanical load, which liquefies under the influence of mechanical shearing forces Fr, such as, for example, during extrusion through a nozzle 7 or cannula, and, after leaving the nozzle 7, re-solidifies due to the self-healing properties and the reversible crosslink 14. The hydrogel material according to the invention can thus be applied to a substrate 8 by extrusion through a nozzle 7, whereby a complex three-dimensional solid body can be formed by applying a generative method, such as, for example, 3D printing. The arrows represented in the nozzle 7 illustrate the shear force effect on the hydrogel material located in the nozzle 7. The shear force Fr acting on the hydrogel material increases in the direction of the nozzle outlet, as a result of which previously formed crosslinks 14 between the main chain polymers 1 according to the enlargement 5 shown in FIG. 1 are dissolved again. The already solidified hydrogel material is liquefied again by the influence of shear forces and can be applied as drops to the substrate 8. After application, the hydrogel material can be relaxed on the substrate 8, so that the crosslinks 14 are restored and the hydrogel material solidifies again. The hydrogel material according to the invention can thus be employed as a bio ink.

FIG. 3 shows a schematic representation for explaining the structure of the main chain polymers 1 of the hydrogel material according to the invention. The basic structure of acryloyl-based monomers 9, which can be synthesised by free radical chain reaction, is shown. An arrow 13 shows an example of a main chain polymer 1 formed from the monomers 9 with the anchor modules 2 and peptide side chains 10. The monomers 9 are derivatives of acrylamide and acrylic acid. These monomers 9 can be present in different mixtures and in any desired ratios. By using small amounts of initiator, such as ammonium persulphate, molecular weights of the main chain polymers 1 of 100 kDa to 50 MDa can be achieved under anaerobic conditions. The anchor modules 2 and the peptide side chains 10 are bonded to the main chain polymers 1 via amide bindings. The individual monomers 9 can have the following substituents at the positions R1 and R2:
R1: amino group, hydroxy group, methoxy group, O-(2-aminoethyl)-O′-methyl polythene glycol group, 3-hydroxypropane-1-sulfonic acid group, isopropylamino group
R2: hydrogen or methyl group

FIG. 4 shows a further schematic representation for explaining the structure of the hydrogel material according to the invention. On the left side, a single main chain polymer 1 with the anchor modules 2 modified thereon is shown in the form of DNA single strands and peptide side chains 10. On the right side, three main chain polymers 1 are shown by way of example within the matrix of the hydrogel material. An arrow 15 symbolises the influence of DNA modules 4 in the form of free DNA single strands within the matrix of the hydrogel material in which the main chain polymers 1 are contained. The DNA modules 4 are a constituent of the hydrogel material and serve primarily to form the crosslinks 14 between the main chain polymers 1 as a result of intermolecular DNA double strand formation. In addition, the DNA modules 4 can have different further functionalisations in order to perform different functions with the anchor modules 2 of the main chain polymers 1. The DNA modules 4 consist of synthetic DNA single strands which can be formed with or without covalent modification. These DNA modules 4 have the property of integrating into the hydrogel material according to the invention by molecular self-assembly and thus imparting specific properties to the hydrogel material in interaction with the anchor modules 2.

FIG. 5 shows a schematic representation for explaining the crosslinking of the hydrogel material using DNA modules 4 from a provided crosslinker library, CL. A CL contains a specific number of DNA modules 4 in the form of predetermined DNA single strand pairs, which can be referred to as splint pairs 11. The splint pairs 11 formed from two DNA single strands 11.1 and 11.2 in each case have specific sequences in order to suppress intramolecular connections 12 between anchor modules 2 of the same main chain polymer 1.

The splint pairs 11 have a common binding domain 16 at which the DNA single strands form a DNA double strand, wherein each DNA single strand of a splint pair 11 has an identical anchor module binding domain 17 which binds to the anchor module 2 of a main chain polymer 1 and forms an intermolecular DNA double strand. The common binding domain 16 of a splint pair 11 is formed in such a way that in each case only the two partner DNA single strands of the splint pair 11 bind to one another in a predetermined sequence region. In doing so, the common binding domains 16 can be such that their sequences are orthogonal to one another, i.e. the sequences are unique and optimised for maximum binding specificity. Different splint pairs 11 in the form of DNA single strand pairs are available, which can be selected from the library CL containing DNA single strand pairs with a different number of binding pairs within the common binding domain 16.

FIG. 6 shows a further schematic representation for explaining the structure of combinatorial splint pair libraries (CCLs) which can be employed as DNA modules 4 for crosslinking the main chain polymers 1 in the hydrogel material. The CCL contains DNA single strand pairs, i.e. splint pairs 11, which differ in that the DNA sequence of the common binding domain 16 has a predetermined number n of different base combinations at specific sequence positions N. The combinatorial crosslinker libraries CCL resulting from this approach can have a very high number of splint pairs 11. The complexity, i.e. the number of different splint pairs 11 within a CCL, depends on the number of sequence positions N within the common binding domain 16. A corresponding example of such a library is shown in FIG. 6. On the left side under the letter a), a DNA module 4 in the form of a splint pair 11 is represented by way of example and greatly simplified. The splint pair 11 has two DNA single strands 11.1 and 11.2 which are connected to one another at a common binding domain 16 and form a DNA double strand. The dashed line represents an enlargement of the region of the binding domain 16, wherein the complementary bases of the DNA single strands 11.1 and 11.2 are represented by the letter B for the sake of simplicity. The sequence of the common binding domain 16 has no sequence positions N with different base combinations, so that exactly one splint pair 11 is available for this sequence configuration. Both DNA single strands 11.1 and 11.2 have an anchor module binding domain 17. A CCL is represented under the letter b), which has four spline pairs 11. The number of splint pairs 11 of this CCL results from the possibility of combining the bases of adenine (A), thymine (T), guanine (G) and cytosine (C) at the sequence position N within the sequence. At the sequence position N, the base combinations A-T, T-A, C-G and G-C are thus available. The letter c) of FIG. 6 represents a CCL in which the DNA sequence of the common binding domain 16 of the splint pairs 11 has a base combination possibility at two sequence positions N. As a result of the combination possibility of the bases of adenine (A), thymine (T), guanine (G) and cytosine (C) at the sequence positions N of the binding domain 16, sixteen splint pairs 11 result as a constituent of this CCL.

The binding specificity of the common binding domain 16 of splint pairs 11 can be extended or varied in a combinatorial manner by a fixed number n of bases N. The number of different binding pairs of the splint pairs 11 of a CCL is 4ⁿ according to the example shown in FIG. 6. Due to the introduction of mixed base pairs at specific sequence positions N of the splint pairs 11, the dissociation temperature or the melting temperature at the common binding domain 16 can be varied. This ensures that, for example, in the case of a slow cooling from a temperature of 80° C., the splints 11.1, 11.2 first bind with their anchor module binding domain 17 to an anchor module 2 at a first melting temperature T1. As soon as the temperature reaches a lower melting point T2, the respective DNA single strands of the splint pairs 11 connect with their suitable partner.

FIG. 7 shows a schematic representation for explaining the influence of temperature-dependent DNA blocking strands 18, as well as the experimentally observed effect of temperature-dependent DNA blocking strands 18 on the memory module G′. The temperature-dependent DNA blocking strands 18 are DNA single strands which block the common binding domain 16 of the splint pairs 11 below the melting temperature of the common binding domain 16 of splint pairs 11. The DNA sequence of the blocking strands 18 is selected such that they spontaneously dissociate at a predetermined temperature, for example 15° C., thereby releasing the binding site for the common binding domain 16 of the splint pairs 11. In this way, the splint pairs 11 can be introduced into the hydrogel material according to the invention as DNA modules 4 with a blocked common binding domain 16, wherein crosslinking leads to crosslinking of the anchor modules 2 only by increasing the temperature of the hydrogel material above 15° C., in that the common binding domains 16 of the splint pairs 11 are released by release of the blocking strands 18.

FIG. 8 shows a schematic representation for explaining the functioning of DNA modules 4 with the property of forming a hairpin secondary structure 19 or hairpin loop structure 19, which is employed for self-blocking of the common binding domains 16. In this embodiment variant, the DNA modules 4 are in the form of splint pairs 11, wherein the presence of the self-blocking hairpin secondary structure 19, which is respectively formed on the DNA single strands 11.1 and 11.2 in the region of the common binding domain, leads to slower crosslinking kinetics. The spontaneous opening of the hairpin secondary structures 19 can be accelerated by an increase in temperature, as a result of which the common binding domain 16 of the relevant splint pair 11 is released and thus the desired crosslinking can occur. Compared to the control shown in FIG. 7 over the reaction kinetics using DNA blocking strands 18, the hairpin secondary structures 19 shown here do not release double strand DNA by the intramolecular binding.

### LIST OF REFERENCE NUMERALS

1 main chain polymer
2 anchor module
3 enlargement
4 DNA module
5 enlargement
6 enlargement
7 nozzle
8 substrate
9 monomer
10 peptide side chain
11 DNA single strand pair/splint pair
11.1 splint/DNA single strand
11.2 splint/DNA single strand
12 intramolecular compound
13 arrow
14 crosslink
15 arrow
16 common binding domain
17 anchor module binding domain
18 DNA blocking strand
19 hairpin secondary structure/hairpin loop structure

1. A modifiable hydrogel material comprises:
main chain polymers modified with predetermined anchor modules in the form of predetermined functionalised DNA single strands,
wherein the main chain polymers are crosslinked with one another by intermolecular DNA double strand formation,
wherein a DNA sequence of the anchor modules has a predetermined number of specific sequence positions N with different base combinations and/or is blocked by a temperature-dependent DNA-blocking strand.

2. The modifiable hydrogel material according to claim 1, wherein the main chain polymers each have a molecular weight in the range of 100 kDa and 50 MDa.

3. The modifiable hydrogel material according to claim 1, wherein the main chain polymers are formed from acryloyl-based monomers.

4. The modifiable hydrogel material according to claim 1, wherein the main chain polymers are modified with peptide side chains.

5. The modifiable hydrogel material according to claim 1, wherein the DNA single strands of the anchor modules have covalent modifications.

6. The modifiable hydrogel material according to claim 1, wherein the main chain polymers have peptide side chains which contain at least one of an RGD sequence or an IKVAV sequence.

7. The modifiable hydrogel material according claim 1, having DNA modules in the form of free DNA single strands.

8. The modifiable hydrogel material according to claim 7, wherein the DNA modules have a predetermined anchor module binding domain.

9. The modifiable hydrogel material according to claim 8, wherein the DNA modules are formed in predetermined DNA single strand pairs which form an intermolecular DNA double strand on a common binding domain, wherein each DNA single strand has an identical anchor module binding domain which binds to the anchor module of a main chain polymer and forms an intermolecular DNA double strand, wherein several different DNA single strand pairs differ in the sequence of the common binding domain.

10. (canceled)

11. The modifiable hydrogel material according to claim 9, wherein the DNA sequence of the common binding domain has a predetermined number n of specific sequence positions N with different base combinations, wherein the number n is in the range from 0 to 5.

12. The modifiable hydrogel material according to claim 9, wherein the anchor module binding domain and the common binding domain have different melting temperatures, wherein the melting temperature of the anchor module binding domain is higher than the melting temperature of the common binding domain.

13. The modifiable hydrogel material according to claim 8, wherein the DNA modules are blocked by a temperature-dependent DNA blocking strand.

14. The modifiable hydrogel material according to claim 13, wherein the temperature-dependent DNA blocking strand has a DNA sequence which dissociates at a temperature in the range from 4° C. to 37° C.

15. The modifiable hydrogel material according to claim 8, wherein at least one of the anchor modules or the DNA modules includes a structure-forming DNA sequence which is self-blocking below a predetermined dissociation temperature while forming a hairpin loop structure.

16. The modifiable hydrogel material according to claim 9, wherein a stress relaxation behaviour can be set by the base sequence and the sequence length of the anchor module binding domain and/or by the base sequence and the sequence length of the common binding domain.

17. The modifiable hydrogel material according to claim 16, wherein the sequence of at least one of the anchor module binding domain or the common binding domain includes 8 to 22 nucleotides.

18. The modifiable hydrogel material according to claim 1, including bait DNA in the form of free predetermined synthetic DNA single strands.

19. The modifiable hydrogel material according to claim 1, including a predetermined proportion of the protein actin and/or a predetermined proportion of a chelator.

20. The modifiable hydrogel material according to claim 8, wherein the anchor modules and/or the DNA modules have at least one modified DNA domain with a functionalisation as a DNA switch, DNA sensor, DNA-enzymatic actuator and/or aptamer.

21. A method for producing a hydrogel with a hydrogel material having main chain polymers which are modified with anchor modules in the form of predetermined functionalised DNA single strands, comprising crosslinking the main chain polymers with one another by intermolecular DNA double strand formation, wherein DNA modules in the form of free DNA single strands or DNA modules in the form of predetermined DNA single strand pairs which bind to the anchor modules in a complementary manner are used for crosslinking the main chain polymers, wherein the free DNA single strands or DNA modules at a common binding domain form an intermolecular DNA double strand, wherein each DNA single strand has an identical anchor module binding domain which binds to the anchor module of a main chain polymer and forms an intermolecular DNA double strand.

22.-27. (canceled)
